(19) United States
(12) Patent Application Publication
(10) Pub. No.: US 2019/0269579 A1
(43) Pub. Date: Sep. 5, 2019

(54) Dental Varnish

(71) Applicant: Toothshine Limited, London (GB)

(72) Inventors: Sarah Hafner, London (GB); Michael Hafner, Manchester (GB); William Mark Palin, Birmingham (GB); Mohammed Abdul Hadis, Birmingham (GB)

(21) Appl. No.: 16/290,221

(22) Filed: Mar. 1, 2019

(30) Foreign Application Priority Data
Mar. 1, 2018 (GB) 1803368.8

(51) Int. Cl. A61K 6/00; A61K 8/25; A61K 8/29; A61K 8/28

(52) U.S. Cl. CPC A61K 6/0017; A61K 8/25; A61K 8/26; A61K 8/28; A61K 8/29

(57) ABSTRACT

Provided is a dental varnish composition comprising: a) a resin composition comprising: (i) an adhesive resin prepared from: one or more acidic monomers selected from 4-META, PMGDM, HEMA-phosphate, NTG-GMA, or salts thereof; and MDP; ii) a base resin prepared from: a high molecular weight monomer selected from Bis-GMA, UDMA, Bis-EMA or combinations thereof; and a low molecular weight monomer selected from TEGDMA, MMA, PEGDMA or combinations thereof; iii) a solvent; iv) a photoinitiator; v) HEMA or salts thereof; c) pigment; and d) optionally a filler.

### CROSS-REFERENCE TO RELATED APPLICATION

This application claims the benefit of priority of Great Britain Patent Application No. 1803368.8, filed on Mar. 1, 2018, the entire contents of which are incorporated by reference herein.

### FIELD OF THE INVENTION

The present invention relates to a dental varnish. The dental varnish is semi permanent, and can be removed and reapplied. The dental varnish changes the colour and appearance of teeth. The present invention also relates to a cosmetic method of whitening, colouring and/or changing the appearance of teeth.

### BACKGROUND TO THE INVENTION

Methods of whitening or colouring teeth to improve or change their appearance are well-known. However, such methods can be complex, involving multiple visits to a dental professional and the use of potentially toxic chemicals such as bleaching agents. Furthermore the resulting colour is unpredictable. These treatments can also lead to a permanent change in the appearance of teeth. In some cases it may be desirable for the change in colour or appearance of teeth to only last for a short period of time, for example for a special occasion, and/or for the dental varnish composition to be capable of being removed and reapplied, in much the same way as nail varnish. Home treatments for whitening teeth are also known, but these can be less effective and/or difficult to use.

Known whitening techniques also may not address other imperfections of teeth, such as surface imperfections.

Accordingly, there is a need to provide dental compositions which improve the colour or appearance of teeth in a predictable manner. In particular, there is a need for a semi-permanent dental varnish or composition which can be removed and reapplied. There is also a need for a cosmetic method of whitening, colouring and/or changing the appearance of teeth utilising such a composition.

### SUMMARY OF THE INVENTION

An aspect of the invention provides a dental varnish composition comprising:

a) a resin composition comprising:
(i) an adhesive resin prepared from:
one or more acidic monomers selected from 4-methacryloyloxyethyl trimellitate anhydride (4-META), pyromellitic dimethacrylate (PMGDM), 2-hydroxyethylmethacrylate acid phosphate (HEMA-phosphate), N-tolylglycine glycidyl methacrylate (NTG-GMA), or salts thereof, wherein the total amount of the one or more acidic monomers is from 0.5 to 25 wt % of the resin composition; and
10-methacryloyloxydecyl dihydrogen phosphate (MDP) in an amount of 0.5 to 20 wt % of the resin composition
ii) a base resin prepared from:
a high molecular weight monomer selected from bisphenol A glycidyl methacrylate (Bis-GMA), urethane dimethacrylate (UDMA), Ethoxylated Bis Phenol A Dimethacrylate (Bis-EMA) or combinations thereof in an amount of from 15 to 30 wt % of the resin composition; and
a low molecular weight monomer selected from triethylene glycol dimethacrylate (TEGDMA), methyl methacrylate (MMA), poly(ethylene glycol) dimethacrylate (PEGDMA) or combinations thereof in an amount of from 0.5 to 15 wt % of the resin composition;
iii) a solvent comprising one or more alcohols or one or more ketones or combinations thereof in an amount of 15-50 wt % of the resin composition;
iv) a photoinitiator in an amount of from 0.1 to 2 wt % of the resin composition;
v) 2-hydroxyethylmethacrylate (HEMA) or salts thereof in an amount of from 5 to 20 wt % of the resin composition;
b) pigment in an amount of from 0.1 to 20 wt % of the varnish composition; and
c) optionally a filler in an amount of from 1 to 20 wt % of the varnish composition.

Many of the components of this composition are known for use as restorative materials in dentistry, in particular to fill dental cavities. However, the composition of the invention is not known for use as a varnish on the surface of teeth to change the colour or appearance of teeth. It has been surprisingly found that the above composition can be used to prepare a varnish, typically a semi permanent varnish for teeth to improve or change the colour or cosmetic appearance of teeth. A great advantage of the composition of the invention is that it can be applied to teeth, removed and reapplied to teeth on a regular basis to predictably change the appearance of teeth; in much the same way as nail varnish can be used to change the appearance of nails. Compositions which can be applied to the surface of teeth to change the appearance of teeth are known; however these compositions can not be easily removed and reapplied in contrast to the varnish compositions of the present invention.

### BRIEF DESCRIPTION OF THE DRAWINGS

FIG. 1 shows the degree of conversion of formulations listed in Table 1.

FIG. 2 shows the bond strength of materials reported in Table 1 following aqueous immersion.

### DETAILED DESCRIPTION

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

Each individual component of the dental varnish compositions of the invention is commercially available, for example:

### 4-META: Polysciences, Inc

### MDP: Watson International

### PMGDM: Esschem

### NTG-GMA: Esschem

### Bis-GMA: Sigma Aldrich

### Bis-EMA: Sigma Aldrich

### UDMA: Sigma Aldrich

### PEGDMA: Esschem

### TEGDMA: Sigma Aldrich

### MMA: Sigma Aldrich

### HEMA: Sigma Aldrich

### TPO: Sigma Aldrich

### CQ: Sigma Aldrich

### DMAEMA: Sigma Aldrich

### DPI: Sigma Aldrich

The dental varnish composition comprises a resin. The resin comprises a co-monomer mixture of an adhesive resin and a base resin.

On exposure to light the photoinitiator is activated, forming free radicals which initiate the polymerisation and/or copolymerisation of the monomers to form the resin.

The adhesive resin enables the composition to adhere or attach to the tooth enamel surface. The adhesive resin is prepared from one or more acidic monomers selected from 4-methacryloyloxyethyl trimellitate anhydride (4-META), pyromellitic dimethacrylate (PMGDM), 2-hydroxyethylmethacrylate acid phosphate (HEMA-phosphate), N-tolylglycine glycidyl methacrylate (NTG-GMA), or salts thereof, wherein the total amount of the one or more acidic monomers is from 0.5 to 25 wt % of the resin composition; and 10-methacryloyloxydecyl dihydrogen phosphate (MDP) in an amount of 0.5 to 20 wt % of the resin composition.

The total amount of the one or more acidic monomers is from 0.5 to 25 wt % of the resin composition, optionally 5 to 20 wt %, and preferably 2 to 6 wt %.

The amount of MDP in the resin composition is from 0.5 to 20 wt %, optionally 10 to 20 wt %, and preferably 15 to 20 wt %.

In a particularly preferred embodiment, the adhesive resin is prepared using 4-META and MDP. MDP is also an acidic monomer compound. The monomers are mixed and polymerised to form the adhesive resin.

The base resin is prepared from:

a high molecular weight monomer selected from bisphenol A glycidyl methacrylate (Bis-GMA), urethane dimethacrylate (UDMA), Ethoxylated Bis Phenol A Dimethacrylate (Bis-EMA) or combinations thereof in an amount of from 15 to 30 wt % of the resin composition; and
a low molecular weight monomer selected from triethylene glycol dimethacrylate (TEGDMA), methyl methacrylate (MMA), poly(ethylene glycol) dimethacrylate (PEGDMA) or combinations thereof in an amount of from 0.5 to 15 wt % of the resin composition.

The high molecular weight monomer is present in an amount of from 15 to 30 wt % of the resin composition, preferably 20 to 30 wt %.

The low molecular weight monomer is present in an amount of from 0.5 to 15 wt % of the resin composition, optionally 2 to 6 wt %.

The high molecular weight monomer provides structural and mechanical support to the base resin. This monomer typically provides a polymer which is tacky or adhesive in nature. The low molecular weight monomer is provided to reduce the viscosity of the resin and increase crosslinking, which may adjust the service life of the varnish. The high molecular weight monomer(s) and low molecular weight monomer(s) undergo co-polymerisation to form a base resin with rigidity and strength.

Preferably the acidic monomer is 4-META:

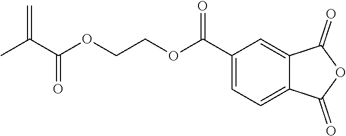 [SMILES: C=C(C)C(=O)OCCOC(=O)c1ccc2c(c1)C(=O)OC2=O]

MDP has the structure:

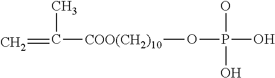

Preferably the high molecular weight monomer is bis-GMA:

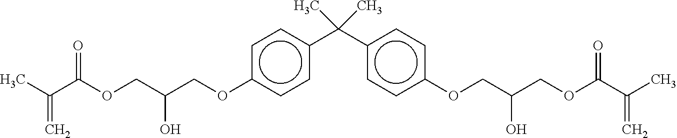

Preferably the low molecular weight monomer is TEGDMA:

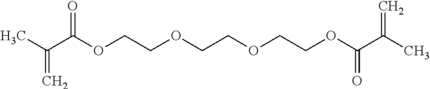 [SMILES: C=C(C)C(=O)OCCOCCOCCOC(=O)C(=C)C]

The resin composition comprises a solvent. The solvent comprises one or more alcohols or one or more ketones or combinations thereof in an amount of 15-50 wt % of the resin composition. Typically the solvent comprises acetone, ethanol or combinations thereof. The solvent preferably comprises water. Typically the acetone or ethanol is 25-75% acetone or ethanol. Most preferably the solvent comprises acetone and water. Preferably, the solvent is present in an amount of from 28 to 40 wt % of the resin composition, optionally 14 to 20 wt % water and 14 to 20 wt % alcohol or ketone, most preferably 18 wt % water and 18 wt % alcohol or ketone.

The dental varnish composition comprises a photoinitiator. Photoinitiators used in the present invention are commercially available.

The photoinitiator absorbs light of a particular wavelength. In the present invention a light source having a wavelength of from 380 nm to 550 nm, preferably 400 to 410 nm is used. The light source is provided by a commercially available light curing machine, for example a Bluephase Style curing light from Ivoclar adent Inc.

Typically the photoinitiator is camphoroquinone (CQ) in combination with a co-initiator, trimethylbenzoylphenyl phosphate (TPO), monacylphosphine oxide, bisacylphosphine oxide, or a benzoyl germanium derivative and the composition optionally further comprises a photosensitizer. The benzoyl germanium derivative may be benzoyltrimethylgermane or dibenzoyldiethylgermane.

In one embodiment the co-initiator is a tertiary amine. The co-initiator may be dimethylaminoethyl methacrylate (DMAEMA), ethyl-4-dimethylaminobenzoate, or a benzodioxole derivative. In a preferred embodiment the co-initiator is dimethylaminoethyl methacrylate (DMAEMA). In a particularly preferred embodiment a photosensitizer is present. Typically the photosensitizer is diphenyliodonium salts.

In one embodiment the photoinitiator is CQ and the co-initiator is DMAEMA. In one embodiment the photoinitiator is TPO.

These photoiniators are selected because they are not significantly affected by the acidic conditions of the varnish resin.

On exposure to light, photocatalysis of the initiator and co-initiator compounds forms radicals which co-polymerise the monomers simultaneously. Typically, the methacrylate groups of the monomers such as bisGMA, TEGDMA, PEGDMA, MDP react to form crosslinked polymers. The phosphate ester group of the MDP molecule provides chemical adhesion to enamel.

In a preferred embodiment, the dental varnish composition comprises the one or more acidic monomers in an amount of from 2 to 6 wt % of the resin composition, MDP in an amount of 15 to 20 wt % of the resin composition, high molecular weight monomer in an amount of from 20 to 30 wt % of the resin composition, low molecular weight monomer in an amount of from 2 to 6 wt % of the resin composition, the solvent in an amount of from 28 to 40 wt % of the resin composition, the photoinitiator in an amount of from 0.2 to 0.5 wt % of the resin composition, HEMA or salts thereof in an amount of from 10 to 20 wt % of the resin composition, pigment in an amount of from 10 to 20 wt % of the varnish composition; and optionally a filler in an amount of from 5 to 20 wt % of the varnish composition.

The filler may comprise one or more opacifiers. An opacifier is a substance added to a material in order to make the material opaque, for example metal oxides such as titantium dioxide. Opacifiers have a refractive index (RI) substantially different from the other components of the composition. The filler may comprise thixotropic particles. Typically the thixotropic particles have an average diameter of less than 40 nm

Optionally the filler comprises fumed silica, silica, silicate glass, quartz, barium silicate, strontium silicate, barium borosilicate, borosilicate, lithium silicate, amorphous silica, ammoniated or deammoniated calcium phosphate, alumina, zirconia, tin oxide, titanium dioxide, or combinations thereof.

In a preferred embodiment of the dental varnish composition, the filler comprises titanium dioxide, fumed silica, barium borosilicate or combinations thereof. High refractive index strontium or Zirconia based glasses may also be used.

The varnish composition comprises a pigment. Typically, a solution of the pigment is added to a mixture of the resin and filler in a ratio of less than or equal to 1:1 by wt %. By altering the ratio of the amount of pigment to resin/filler mixture, different shades of varnish can be prepared. The pigment may be white, black, red, yellow, light brown, or dark brown. In one embodiment a natural tooth colour is preferred. In another embodiment a white tooth colour is preferred. However, a range of colours is contemplated. For example, it may become fashionable to varnish teeth with brightly coloured varnish. The varnish composition may also have a colour suitable to mask colour imperfections on the tooth surface. The varnish composition may be pearlescent, glittery, or textured. The varnish composition may comprise a texturizing agent.

Typically the pigment comprises a metal oxide. Preferably the metal oxide is titanium dioxide or iron oxide.

Pigmented stock ‘pastes’ may be prepared of, for example, ‘white’, ‘black’, ‘red’, ‘yellow’, ‘light brown’ and ‘dark brown’ pigmentation from various metal oxides. Subsequently a highly pigmented stock solution is made by mixing an aliquot of the prepared filled resin and the pigmented pastes at pre-determined concentrations (<5%). The highly pigmented stock solution may then be supplemented into further aliquots of the non-pigmented filled resin to create various shades of tooth varnish.

The dental varnish composition may be suitable to mask or cover surface texture imperfections. The dental varnish composition may provide a shiny and/or smooth layer on the tooth surface.

Typically the dental varnish composition is a semi-permanent dental varnish composition. This typically means that the dental varnish composition is capable of being removed and reapplied. The dental varnish composition can be removed by mechanical means, for example by chipping off a layer of dental varnish formed on the teeth. The dental varnish composition can also be removed by applying an appropriate orally-acceptable solvent. The solvent can be applied with a brush, microbrush or sponge.

A measure of the semi-permanence of the dental varnish may be the bond strength. In one embodiment, the bond strength of the dental varnish composition after application of the varnish composition to tooth enamel and immersion of the varnished tooth enamel in aqueous solution at 37° for a period of 2 weeks is from 5-20 MPa, optionally 8-20 MPa, optionally 10-MPa. The bond strength of the tooth varnishes were typically assessed using a universal testing machine (Instron 5544) using a previously described shear bond strength test method known to a person skilled in the art.

In a preferred embodiment, the dental varnish composition further comprises a film-forming agent. This improves the formation of the layer of varnish of the tooth enamel. In particularly preferred embodiments the film-forming agent is collodion or an adipic acid-neopentyl glycol-trimellitic anhydride copolymer.

In an additional embodiment, the composition further comprises an agent suitable for releasing metal or inorganic ions. In one embodiment the metal or inorganic ions are calcium ions, phosphate ions or fluoride ions. In a preferred embodiment the agent suitable for releasing metal ions is a bioactive glass. In one embodiment, the bioactive glass has substantially similar optical properties to the resin. By substantially similar optical properties is typically meant a similar refractive index, for example a 5-20% difference between the refractive index of the bioactive glass and the refractive index of the resin. In a preferred embodiment the bioactive glass is a calcium silica glass.

In a preferred embodiment the dental varnish composition does not comprise bleach or standard bleaching agents. Bleach/bleaching agents can be toxic or damaging to teeth.

In a further aspect, the invention provides a cosmetic method of whitening, colouring and/or changing the appearance of teeth, the method comprising applying a dental varnish composition as defined above to the tooth enamel of a subject, and curing the varnish with light having a wavelength in the range of from 380 nm to 550 nm, optionally 400 to 410 nm.

In a preferred embodiment of the method, the tooth enamel is cleaned and air dried prior to applying the dental varnish. However, typically the tooth surface should not be completely dried or desiccated.

In one embodiment of the method, after curing the varnish any excess varnish composition is removed.

The varnish composition may be applied as a single layer or phase. Optionally, the varnish forms two or more layers or phases. For example, the composition defined in claim 1 may comprise the base layer, and a top coat layer may be formed on top of the base layer,

In an embodiment of the method, a top coat composition is applied after the curing step, optionally wherein the top coat composition forms a transparent layer.

Typically, the dental varnish composition and/or the top coat composition are applied with a brush or microbrush.

In one embodiment, the method further comprises the step of mechanically removing the varnish. The varnish is typically removed when whitening or colouring of the teeth is no longer required, or when the appearance of the varnish has been affected by chipping or discolouration.

Various preferred features and embodiments of the present invention will now be described by way of non-limiting examples.

### EXAMPLES

### Example 1—Resin Preparation

Compositions are all based on weight %. All materials except material G were subsequently loaded with 5% fumed silica filler and 5% titanium dioxide pigment. Material G was loaded with 10% fumed silica filler and 5% titanium dioxide pigment.

Material A was modified to improve handling and adhesion as follows:

Material B—solvent increased to 50% of resin composition: this compromised handling properties and polymerisation

Material C—acidic monomer removed to act as control

Material D—MDP completely replaced with 4-META

Material E—MDP partially replaced with 4-META (80% MDP, 20% 4-META)

Material F—Resin Matrix modified to 95% Bis-GMA and 5% TEGDMA

Material G—Nano filler content in Material E increased from 5% to 10%.

TABLE 1

| | COMPOSITION % | | | | | | |
|---|---|---|---|---|---|---|---|
| Material | A | B | C (control) | D | E | F | G |
| 4-META | 0 | 0 | 0 | 20 | 4 | 4 | 4 |
| MDP | 20 | 20 | 0 | 0 | 16 | 16 | 16 |
| Bis-GMA | 22.86 | 11.66 | 38.86 | 22.86 | 22.86 | 27.15 | 22.86 |
| TEGDMA | 5.72 | 2.92 | 9.72 | 5.72 | 5.72 | 1.43 | 5.72 |
| ACETONE | 18 | 33.35 | 18 | 18 | 18 | 18 | 18 |
| Water | 18 | 16.55 | 18 | 18 | 18 | 18 | 18 |
| HEMA | 15 | 15 | 15 | 15 | 15 | 15 | 15 |
| TPO | 0.42 | 0.42 | 0.42 | 0.42 | 0.42 | 0.42 | 0.42 |

### Example 2—Varnish Compositions

After preparing resin compositions for the varnish, example varnish compositions were prepared. Compositions of the resin, fillers, film forming agent and pigment stock are set out in Tables 2 to 5 respectively.

TABLE 2
Varnish components (Resin)

| | | Molecular | Density | | | Individual | | |
|---|---|---|---|---|---|---|---|---|
| Group | Component | Weight | (g/cm3) | Target % | Composition | composition % | Range | Alternatives |
| A | 4-META | 304.2 | | 20.00 | 20% | 4 | <10% | PMGDM, HEMA |
| | | | | | | | | phosphate, NTG- |
| | | | | | | | | GMA Magnesium |
| | | | | | | | | salt, surface active |
| | | | | | | | | sodium salts |
| A | MDP | 322.33 | | | 80% | 16 | <20% | None |
| B | Bis-GMA | 512.59 | 1.161 | 28.58 | 85% | 24.293 | 80-95% | UDMA, Bis-EMA |
| B | TEGDMA | 286.32 | 1.079 | | 15% | 4.287 | <20% | MMA, PEGDMA, |
| | | | | | | | | and other |
| | | | | | | | | difuctional |
| | | | | | | | | methacrylate |
| | | | | | | | | monomers |
| C | ACETONE | 58.08 | 0.791 | 36.00 | 50% | 18 | <20% | Ethanol or similar |
| | | | | | | | | solvent |
| C | Water | 18.02 | 1 | | 50% | 18 | <20% | None |
| D | HEMA | 130.14 | 1.073 | 15.00 | | 15 | <20% | HEMA phosphate |
| E | TPO | 418.86 | | 0.42 | | 0.42 | <0.5% | BAPO (Irgacure) |

TABLE 3
Varnish components (Fillers) - these are added to resin mixture

| | | Refractive | | | | |
|---|---|---|---|---|---|---|
| Group | Component | index | Target % | Composition | Range | Alternatives |
| A | Titanium Dioxide | | 10.00 | 50% | <5% | other opacifiers (oxides) |
| A | 14 nm Fumed Silica | | | 50% | <10% | Other thixotropic particles (<40 nm) |
| B | G018-159 Barium | 1.61 | 5.00 | 100% | <10% | Other high RI strontium or Zirconia |
| | filler | | | | | based glasses |

TABLE 4
Varnish components (film formers) - these
are added to resin mixture/filler mixture

| | Compo- | Target | Compo- | | |
|---|---|---|---|---|---|
| Group | nent | % | sition | Range | Alternatives |
| A | Collodion | 10% | 100% | <20% | Adipic acid/ |
| | | | | | Neopentyl Glycol, |
| | | | | | Trimellitic Anhydride |
| | | | | | Copolymer |

TABLE 5
Varnish components (Pigments Stock) - these are added to resin/filler/collodion mixture

| Group | Component | Target % | Composition | Range | Alternatives | Commentary |
|---|---|---|---|---|---|---|
| A | Non-Pigmented Resin/filler | 85.00 | 100.00000% | >50% | N/A | Pigmented stock |
| B | White | 15.00 | 7.79376 | <15% | Other Metal Oxides | solution can be added |
| B | Black | | 4.25567 | <10% | Other Metal Oxides | to the resin/filler |
| B | Red | | 2.12784 | <10% | Other Metal Oxides | mixtures (upto a 1:1 |
| B | Yellow | | 42.55672 | >30% | Other Metal Oxides | ratio) to obtain various |
| B | Light Brown | | 42.55672 | >30% | Other Metal Oxides | shades of varnishes |
| B | Dark Brown | | 0.01538 | <5% | Other Metal Oxides | |

### Example 3—Method of Preparing Varnish Composition

The resin matrix was formulated at 60° C. on a magnetic stirrer hot plate by mixing the base resin and diluent resin for 1 hr to obtain a homogenous mixture. Following the resin matrix preparation, the acidic components, solvent and the photoinitiator components were added and stirred for a further 30 minutes. All reagents were used as received (commercially obtained) except the solvents which were diluted to the desired concentration. Following resin preparation, inorganic particles which included fillers and opacifiers were loaded into the resin. The filled resins were then optionally supplemented with film formers (Collodion). Pigmented stock ‘pastes’ were prepared of, for example, ‘white’, ‘black’, ‘red’, ‘yellow’, ‘light brown’ and ‘dark brown’ pigmentation. Subsequently a highly pigmented stock solution was made by mixing an aliquot of the prepared filled resin and the pigmented pastes at pre-determined ratios. The highly pigmented stock solution was then supplemented into further aliquots of the non-pigmented filled resin to create various shades of tooth varnish.

### Example 4—Application and Light Curing

The material is applied using a microbrush or similar dental apparatus directly onto lightly surface dried enamel. The material is then agitated over the surface for approximately 30 s and lightly air dried to evaporate the solvent. An homogenous coating, no larger than the tip of the light curing unit is recommended. The material is then irradiated for 30 s at a minimum of 1000 mW/cm² either with a commercial dual peak LED light source currently available, or a bespoke light source with peak spectral output between 400-410 nm. Photocatalysis of the initiator and co-initiator compounds form radicals which co-polymerise functional methacrylate groups of each monomer simultaneously, e.g. crosslinking between the functional groups of bisGMA, TEGDMA, PEGDMA, MDP. The dihydrogen phosphate ester group of the MDP molecule provide chemical adhesion to enamel by etching the surface and forming chemical bonds with calcium and hydroxy apatite.

### Example 5—Degree of Conversion

The varnish compositions were tested to see how well the resin cures on application of light. This was determined by measuring the percentage conversion of the resin monomers.

Degree of conversion was assessed in real time using Fourier Transform Near infra-red using an attenuated total reflectance (ATR) method. The spectrometer was fitted with a liquid nitrogen cooled MCT detector to improve the signal to noise ratio whilst maintaining high acquisition rates. Degree of conversion was assessed by measuring the decrease in the intensity of the carbon double bond (aliphatic) stretching mode (1637 cm⁻¹). The measurement was based on the peak area ratio between the aliphatic group and an isosbestic point (aromatic: 1608 cm⁻¹) according to equation 1.

$$Conversion \% = \frac{aliphatic \text{/} isosbestic ( peak areas of polymer )}{aliphatic \text{/} isosbestic ( peak areas of monomer )}$$

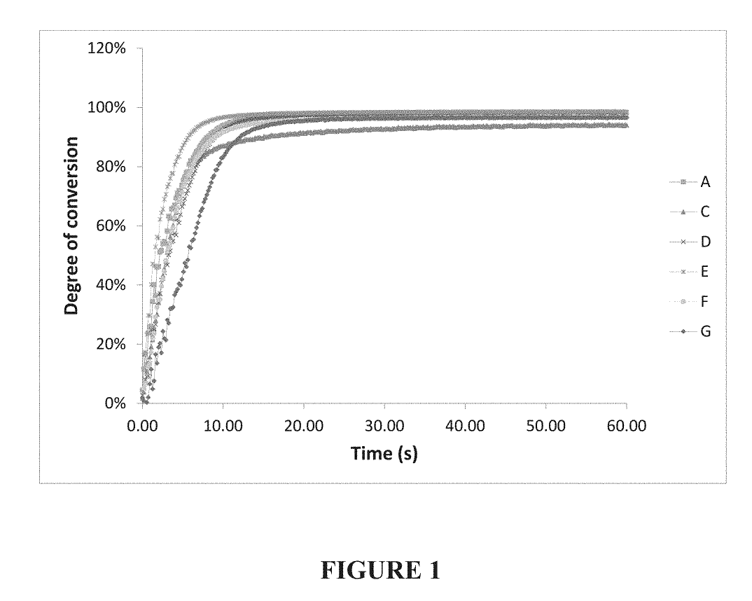

Data was acquired at an acquisition rate of 0.18 s⁻¹ with a resolution of 16 and 2 scans average (data spacing 1.928 cm⁻¹) for 100 s. A silicon mould (8 mm diameter, 0.2 mm thick) was placed centrally over the ATR diamond sensor. The mould was filled with tooth varnish and air dried for 30 s. The varnish was then manipulated to provide a flat surface within the area of the mould. The varnish was then cured for 30 s using a 3^{rd} generation Bluephase Style curing light and DC was measured in real time. Results are shown in FIG. 1.

### Example 6—Bond Strength

Bond strength of the varnish was determined as an indicator of the longevity of the varnish following application of the varnish to the tooth.

Bovine incisors where obtained to provide large enamel surfaces onto which the varnishes could be bonded. The teeth were set in epoxy resin and then polished with sequential grades of silcon carbide paper (P800, P1200 and P2500) using and automated water cooled polishing wheel set to 150 rpm for 5 s at each grade. Care was taken not to expose the dentine whilst obtaining flat enamel surfaces. The samples were then stored in water until used.

A cyclindrical shaped mould was fabricated by sandwiching silicon impression material between two microscope slides and applying pressure to extrude the excess material and leave a thin (˜0.2 mm thick) layer of material sandwiched between the two glass slides. After the impression material had set, the glass slides were removed and an 8 mm diameter hole was punched through the material with a biopsy plunger tool.

Prior to tooth varnish application, the enamel surface were cleaned with 70% ethanol and air dried. The silicon mould was placed centrally over the prepared enamel surfaces leaving an area of 8 mm diameter enamel exposed. Tooth varnish was applied to the exposed enamel surfaces using a microbrush ensuring the varnish did not overfill the mould. The varnish was then rubbed into the enamel using the microbrush for 30 s whilst gently air drying to evaporate off any excess solvent. The varnish was then manipulated to provide a flat surface covering the exposed area. The varnish was then cured for 30 s using a 3^{rd} generation Bluephase Style curing light. A composite stub was then cured centrally onto the tooth varnish surface (4 mm diameter and 2 mm thick, Gradia Direct, A3 shade, 30 s cure) to facilitate bond strength testing.

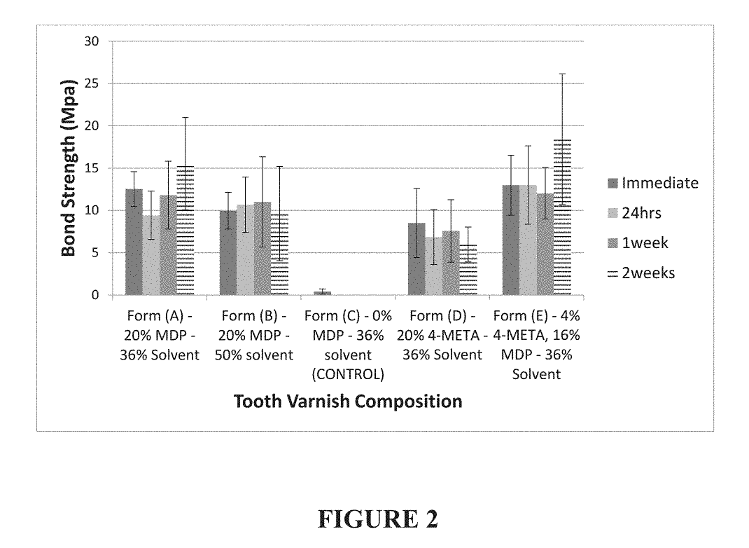

The bond strength of the new tooth varnishes were assessed immediately, 24 hrs, 1 week and 2 weeks following application and aqueous immersion at 37° C. using a universal testing machine (Instron 5544) using the previously described shear bond strength test method. Results are shown in FIG. 2. Formulations A and E exhibited the highest bond strength values and the strength of the bonds were maintained over the two week period. However, the data suggests a combination of acidic monomers is likely to enhance both immediate and longer term bond strength of these materials and thus Material E is preferred.

The present invention is further described by the following claims.

1. A dental varnish composition comprising:
a) a resin composition comprising:
an adhesive resin prepared from:
one or more acidic monomers selected from 4-methacryloyloxyethyl trimellitate anhydride (4-META), pyromellitic dimethacrylate (PMGDM), 2-hydroxyethylmethacrylate acid phosphate (HEMA-phosphate), N-tolylglycine glycidyl methacrylate (NTG-GMA), or salts thereof, wherein the total amount of the one or more acidic monomers is from 0.5 to 25 wt % of the resin composition; and
10-methacryloyloxydecyl dihydrogen phosphate (MDP) in an amount of 0.5 to 20 wt % of the resin composition
ii) a base resin prepared from:
a high molecular weight monomer selected from bisphenol A glycidyl methacrylate (Bis-GMA), urethane dimethacrylate (UDMA), Ethoxylated Bis Phenol A Dimethacrylate (Bis-EMA) or combinations thereof in an amount of from 15 to 30 wt % of the resin composition; and
a low molecular weight monomer selected from triethylene glycol dimethacrylate (TEGDMA), methyl methacrylate (MMA), poly(ethylene glycol) dimethacrylate (PEGDMA) or combinations thereof in an amount of from 0.5 to 15 wt % of the resin composition;
iii) a solvent comprising one or more alcohols or one or more ketones or combinations thereof in an amount of 15-50 wt % of the resin composition;
iv) a photoinitiator in an amount of from 0.1 to 2 wt % of the resin composition;
v) 2-hydroxyethylmethacrylate (HEMA) or salts thereof in an amount of from 5 to 20 wt % of the resin composition;
b) pigment in an amount of from 0.1 to 20 wt % of the varnish composition; and
c) optionally a filler in an amount of from 1 to 20 wt % of the varnish composition.

2. The dental varnish composition according to claim 1, wherein the solvent comprises acetone, ethanol or combinations thereof, optionally wherein the solvent comprises water.

3. The dental varnish composition according to claim 2, wherein the acetone or ethanol is 25-75% acetone or ethanol.

4. The dental varnish composition according to claim 1, wherein the photoinitiator is camphoroquinone (CQ) in combination with a co-initiator, trimethylbenzoylphenyl phosphate (TPO), monacylphosphine oxide, bisacylphosphine oxide, or a benzoyl germanium derivative and the composition optionally further comprises a photosensitizer.

5. The dental varnish composition according to claim 4, wherein the co-initiator is a tertiary amine.

6. The dental varnish composition according to claim 4, wherein the co-initiator is dimethylaminoethyl methacrylate (DMAEMA), ethyl-4-dimethylaminobenzoate, or a benzodioxole derivative.

7. The dental varnish according to claim 4, wherein the photosensitizer is diphenyliodonium salts.

8. The dental varnish composition according to claim 1, which comprises the one or more acidic monomers in an amount of from 2 to 6 wt % of the resin composition, MDP in an amount of 15 to 20 wt % of the resin composition, high molecular weight monomer in an amount of from 20 to 30 wt % of the resin composition, low molecular weight monomer in an amount of from 2 to 6 wt % of the resin composition, solvent in an amount of from 28 to 40 wt % of the resin composition, a photoinitiator in an amount of from 0.2 to 0.5 wt % of the resin composition, HEMA or salts thereof in an amount of from 10 to 20 wt % of the resin composition, pigment in an amount of from 10 to 20 wt % of the varnish composition; and a filler in an amount of from 5 to 20 wt % of the varnish composition.

9. The dental varnish composition according to claim 1, wherein the filler comprises fumed silica, silica, silicate glass, quartz, barium silicate, strontium silicate, barium borosilicate, borosilicate, lithium silicate, amorphous silica, ammoniated or deammoniated calcium phosphate, alumina, zirconia, tin oxide, or combinations thereof.

10. (canceled)

11. The dental varnish composition according to claim 1, wherein a solution of the pigment is added to a mixture of the resin and filler in a ratio of less than or equal to 1:1 by wt %.

12. The dental varnish composition according to claim 1, wherein the pigment comprises a metal oxide.

13. (canceled)

14. The dental varnish composition according to claim 1, wherein the acidic monomer is 4-META.

15. The dental varnish composition according to claim 1, wherein the high molecular weight monomer is bis-GMA.

16. The dental varnish composition according to claim 1, wherein the low molecular weight monomer is TEGDMA.

17. The dental varnish composition according to claim 1, wherein the photoinitiator absorbs light at a wavelength of 400 to 410 nm.

18. The dental varnish composition according to claim 1, which is a semi-permanent dental varnish composition.

19. The dental varnish composition according to claim 18, wherein the dental varnish composition is capable of being removed by mechanical means or by application of solvent and reapplied.

20. The dental varnish composition according to claim 18, wherein the bond strength of the dental varnish composition after application of the varnish composition to tooth enamel and immersion of the varnished tooth enamel in an aqueous solution for a period of 2 weeks is from 5-20 MPa.

21. The dental varnish composition according to claim 1, further comprising a film-forming agent.

22. (canceled)

23. The dental varnish composition according to claim 1, wherein the composition further comprises an agent suitable for releasing metal or inorganic ions.

24. (canceled)

25. The dental varnish composition according to claim 23, wherein the agent suitable for releasing metal ions is a bioactive glass.

26. (canceled)

27. The dental varnish composition according to claim 25, wherein the bioactive glass is a calcium silica glass.

28. (canceled)

29. (canceled)

30. A cosmetic method of whitening, colouring and/or changing the appearance of teeth, the method comprising applying a dental varnish composition as defined in claim 1 to the tooth enamel of a subject, and curing the varnish with light having a wavelength in the range of from 380 nm to 550 nm, optionally 400 to 410 nm.

31. The cosmetic method according to claim 30, wherein the tooth enamel is cleaned and air dried prior to applying the dental varnish.

32. The cosmetic method according to claim 30, wherein after curing the varnish any excess varnish composition is removed.

33. The cosmetic method according to claim 30, wherein a top coat composition is applied after the curing step, optionally wherein the top coat composition forms a transparent layer.

34. (canceled)

35. The cosmetic method according to claim 33, further comprising the step of mechanically removing the varnish.
